(19) United States
(12) Patent Application Publication
(10) Pub. No.: US 2013/0245505 A1
(43) Pub. Date: Sep. 19, 2013

(54) Noninvasive Ultrasound-Based Retinal Stimulator: Ultrasonic Eye

(71) Applicant: The Board of Trustees of the Leland Stanford Junior University, (US)

(72) Inventors: Butrus T. Khuri-Yakub, Palo Alto, CA (US); Omer Oralkan, Morrisville, NC (US); Stephen A. Baccus, Half Moon Bay, CA (US); Michael D. Menz, San Bruno, CA (US)

(21) Appl. No.: 13/867,588

(22) Filed: Apr. 22, 2013

Related U.S. Application Data
(63) Continuation-in-part of application No. 13/441,650, filed on Apr. 6, 2012, now abandoned.
(60) Provisional application No. 61/516,832, filed on Apr. 8, 2011.
(60) Provisional application No. 61/620,947, filed on Apr. 5, 2012.

(51) Int. Cl. A61F 9/08; A61N 7/00

(52) U.S. Cl. CPC A61F 9/08; A61N 7/00
USPC 601/2

(57) ABSTRACT

A retinal stimulation and prosthetic device is provided that includes at least one ultrasonic transducer having a focused ultrasonic signal, where the focused ultrasonic signal includes an acoustic frequency, a spot size, a temporal pattern, a pulse duration and a power capable of stimulating retinal neurons when the at least one ultrasonic transducer is disposed proximal to an eye.

### CROSS-REFERENCE TO RELATED APPLICATIONS

This application is a continuation-in-part of U.S. patent application Ser. No. 13/441,650 filed Apr. 6, 2012, which is incorporated herein by reference. U.S. patent application Ser. No. 13/441,650 filed Apr. 6, 2012 claims the benefit of U.S. provisional patent application 61/516,832, filed on Apr. 8, 2011, and is hereby incorporated by reference in its entirety. U.S. patent application Ser. No. 13/441,650 filed Apr. 6, 2012 also claims the benefit of U.S. provisional patent application 61/620,947, filed on Apr. 5, 2012, and is hereby incorporated by reference in its entirety.

### STATEMENT OF GOVERNMENT SPONSORED SUPPORT

This invention was made with Government support under contract awarded by The National Institutes of Health (NIH) under grant number R01 EY16842. The Government has certain rights in this invention.

### FIELD OF THE INVENTION

This invention relates to ultrasonic stimulation of neural cells. More specifically, the invention relates to a retinal stimulation and prosthetic device for ultrasound-based noninvasive retinal stimulation for probing remaining retinal function and for restoring sight to the blind.

### BACKGROUND OF THE INVENTION

Cataracts, glaucoma, age-related macular degeneration (AMD), diabetic retinopathy, and retinitis pigmentosa (RP) are some of the leading causes of blindness. In a healthy human eye, vision is accomplished by focusing light onto the retina by a lens. This focused light is then detected by photoreceptor cells in the retina, which stimulates a complex network of neurons in the inner retina. These electrical impulses travel through the optic nerve to the brain and produce vision. Loss of vision can be caused at the optical level by a dysfunctional or blocked lens, at the sensory level by destroyed or degenerated photoreceptor cells, or at the neural level by loss of function of central nervous system tissue. Cataracts, for example, are a clouding of the lenses and cause blurry vision. Glaucoma damages the optic nerve in the eye. AMD destroys the macula, the oval-shaped highly pigmented yellow spot near the center of the retina, and results in loss of central vision. Diabetic retinopathy is a result of microvascular retinal changes caused by complications of diabetes. RP is genetic disorder in which abnormalities of the photoreceptors progressively lead to loss of vision.

Cataracts are highly treatable by replacing the eye's natural lens with an intraocular lens through a surgery. For cases where a selective degeneration of the outer retina impairs vision, there is the potential that a retinal prosthesis can restore sight. For example, in both AMD and RP the photoreceptor cells are significantly degenerated, but the retinal ganglion cells, which are responsible for delivering visual input from the eye to the brain, are relatively spared. Stimulating the nerve cells of the middle and inner retina might provide neural input to the visual cortex that could produce vision. In patients with diabetic retinopathy and glaucoma, the inner retina or optic nerve is damaged. In these cases of neural blindness, restoration of vision would require stimulation of neurons that are postsynaptic to ganglion cells.

There are several companies developing epiretinal and subretinal implants for cases of photoreceptor degeneration. Such an implant has two main components: sensor devices (e.g. miniature camera) to capture the elements of the visual scene and a stimulator (e.g. microelectrode array) to artificially stimulate the nerve tissue. Electrical stimulation is the most common way to drive the nerve cells in these currently available devices. In one system an external camera captures the scene and sends the image data wirelessly to an implanted transistor-based low-power stimulator array. Another approach is to use a photodiode array that is implanted subretinally to capture a visual scene and provide the neural stimulation using the photodiode output current. Another retinal prosthetic system captures the visual scene using an external camera and transmits the image into the eye using a laser beam, which is then captured by an implanted photodiode array. All these approaches need a surgically implanted, biocompatible device. In many cases the implanted device needs to be externally powered using radio-frequency electromagnetic waves. Furthermore, the size and the number of elements in the stimulator array determine the resolution of the image.

Although direct electrical stimulation is the most common technique for stimulating neural cells, other forms of energy are also used. Trans-cranial magnetic stimulation uses electromagnetic induction to induce weak electric currents in the brain using a rapidly changing magnetic field. Genetically targeted neurons within intact neural circuits can be controlled by activation with light. Ultrasound is also known to stimulate neural tissue. The mechanisms of action of ultrasonic neural stimulation may be mechanical or thermal, but the effects are not completely understood. Researchers have recently shown that in the motor cortex of a mouse brain, ultrasound-stimulated neuronal activity was sufficient to evoke motor behaviors.

What is needed is a device and method to stimulate and modulate ongoing neural activity in the retina for the study of circuit function, and used as a noninvasive retinal prosthesis.

### SUMMARY OF THE INVENTION

To address the needs in the art, a retinal stimulation and prosthetic device is provided that includes at least one ultrasonic transducer having a focused ultrasonic signal, where the focused ultrasonic signal includes an acoustic frequency, a spot size, a temporal pattern, a pulse duration and a power capable of stimulating retinal neurons when the at least one ultrasonic transducer is disposed proximal to an eye.

According to one aspect of the invention, the ultrasonic transducer is can be a planar ultrasonic transducer, a planar ultrasonic transducer array, a 2-D flexible disk ultrasonic transducer, a 2-D flexible disk ultrasonic transducer array, an annular ring ultrasonic transducer, of an annular ring ultrasonic transducer array.

In a further aspect of the invention, the acoustic frequency, the spot size, the temporal pattern, the pulse duration and the power are capable of generating response information necessary for evaluating the health of a retina.

According to another aspect of the invention, the focused ultrasonic signal is capable of focusing at any location of a retina.

In yet another aspect of the invention, where the at least one ultrasonic transducer is coupled to an optical imaging system that is capable of imaging a field of view, where the optical imaging system is capable of generating imaging signals capable of exciting the at least one transducer in a manner capable of reproducing an image of the field of view, where the image of the field of view includes a radiation pressure to enabling a sensation of vision.

According to a further aspect of the invention, the acoustic frequency is in a range from 20 MHz to 100 MHz.

In another aspect of the invention, the spot size is in a range of 150 microns to 15 microns.

In another aspect of the invention, the pulse duration is in a range of 0.1 ms to 50 ms.

In another aspect of the invention, the power is in a range of 0.1 to 30 W/cm².

### BRIEF DESCRIPTION OF THE DRAWINGS

FIG. 1 shows a schematic of the experimental setup, according to one embodiment of the invention.

FIG. 2 shows responses of retinal ganglion cells recorded with an array of extracellular electrodes to stimulus onset (ON) and offset (OFF) for a uniform field 0.5 Hz visual flash (data shown in black) and 0.5 Hz ultrasound stimuli, according to one embodiment of the invention.

FIGS. 3a-3c show (3a) left, RMS amplitude of spatio-temporal receptive fields (locations at right), summed over all ganglion cells in a single experiment (arrow to cross indicates ultrasound focus), (3b) change in sensitivity produced by ultrasound stimulus ON (Left) and OFF (Right), (3c) sensitivity as a function of distance from the ultrasound focus during stimulus ON (left) and OFF (right), where the ON gain change shows a center/surround antagonism (resolution has at least as precise as 156 μm and expected resolution from transducer frequency is ˜100 μm), according to one embodiment of the invention.

FIG. 4 shows a block diagram of the ultrasound-based neurostimulation system, according to one embodiment of the invention.

FIGS. 5a-5c show different transducer configurations for acoustic neurostimulation of the retina, according to different embodiments of the invention.

FIGS. 6a-6c show the experimental setup for ultrasound and visual stimulation of the retina, where (6a) shows simulation of the spatial power distribution from the custom ultrasound transducer in the axial (Top, −3 dB width in z=1330 μm) and lateral planes (Bottom, −3 dB width=87 μm), and the scale in dB. (6b) shows a schematic diagram of the ultrasound transducer mounted above the retina and immersed in perfusion fluid while visual stimulation was delivered from below. On the right an expanded view of microscope objective is shown, the ultrasound transducer, and the retina, which was placed ganglion side down on a multielectrode array. (6c) shows a diagram of ultrasound stimulus having a binary temporal modulation of a 43 MHz carrier. The low-frequency modulation was in the range of frequencies used for visual stimuli, according to one embodiment of the invention.

FIGS. 7a-7c show high-frequency modulation of the carrier is no more effective than continuous wave stimulation, where (7a) shows the 43 MHz carrier was modulated at frequencies from 10 Hz to 1 MHz (50% duty cycle), and then modulated again at 0.5 Hz (1 s On, 1 s Off). Other experiments varied the modulation frequency by a factor of 10 spaced at 5 Hz to 500 kHz with similar results (data not shown). (7b) shows raster plots and peristimulus time histogram (PSTH) from two cells. y-axis is the log10 of modulation frequency, where 0 is the continuous wave. The time-averaged power was the same for all conditions (30 W/cm²). For the DC case, there was no additional modulation of the 43 MHz carrier other than 0.5 Hz, and the amplitude was reduced so that the average power was the same for all conditions. Responses at 10 Hz arise from modulation in the range of physiological stimuli. (7c) shows population summary (n=6) of mean firing rate versus modulation frequency. Both On and Off responses were averaged to compute the mean firing rate and then normalized by each cell's maximum mean firing rate, according to embodiments of the invention.

FIGS. 8a-8c show the dependence of response on ultrasound power density, according to one embodiment of the invention.

FIGS. 9a-9d show precise, reproducible activation of the retina from ultrasound stimulation, according to one embodiment of the invention.

FIGS. 10a-5b show ultrasound activates lateral retinal circuitry, where (10a) shows, Top, PSTHs from a single cell at two different locations of the ultrasound transducer. Bottom, Peak firing rate of the On and Off response as a function of distance from the cell's peak location of activation. (10b) shows population summary (n=19) showing the ratio of spatial On response width to the Off response width, according to embodiments of the invention.

FIG. 11a-11e show a comparison of the neural code for ultrasound and visual stimuli (Top) LN models were computed using the standard method of reverse correlation with either the ultrasound stimulus or a 100 μm visual spot, both modulated with binary white noise, where (11a) shows examples of LN models for three cells, for both visual and ultrasound stimuli. (11b) shows latency to first peak of the filter compared for visual and ultrasound stimuli (n=15 from two retinas). Two cells were excluded whose temporal filters in visual and ultrasound stimuli had opposite sign. (11c) shows peak modulation frequency computed from the Fourier transform of the filter. (11d) shows far left. The ultrasound filter from the cell in the top row of (11a) Left middle. The optimal transform filter that transforms the ultrasound filter into the visual filter, Right middle. The transformed ultrasound filter compared with the visual filter (RMS difference between the two filters shown was 8.0%), Far right. All optimal filters that transform the ultrasound filters into the visual filters. The ON cell is identified in the bottom of (11a) shows the cell in the second row of (11a). (11e) shows average sensitivity for each ultrasound and visual condition was computed as the average slope of the nonlinearity. Histogram of ratios of ultrasound to visual sensitivity for each cell (median=1.6, paired t test, p=0.043), according to embodiments of the invention.

FIGS. 12a-12e show how ultrasound rapidly modulates visual responses. (12a) shows visual stimulus was a binary random checkerboard presented simultaneously with a 0.2 s ultrasound pulse delivered every 2 s. Spikes were analyzed relative to the visual stimulus as in Equation 1 but subdivided into three time intervals according to the ultrasound stimulus: On, during the 0.2 s ultrasound stimulus; Off, up to 0.2 s immediately after ultrasound; and Control, 0.1 s after the Off interval until the next On interval. (12b) shows firing rate, temporal filters, and nonlinearities for three cells. Three example cells are shown. (12c) shows changes in threshold and average sensitivity of the nonlinearities caused by ultrasound (n=35). Left, shows change in average sensitivity, computed as the average slope of the nonlinearity, during ultrasound On versus during Off periods. Right, Change in threshold during On period versus Off period. (12d) Left, shows receptive fields of 35 retinal ganglion cells. Arrow-to-cross indicates the ultrasound focus. Middle left, total visual sensitivity across the population. For each cell, sensitivity was computed at each spatial location as the RMS value of the spatiotemporal filter at that spatial location. Total sensitivity was computed by summing across all cells. Middle right, the change in sensitivity produced by ultrasound On computed as the total sensitivity during ultrasound On minus the total sensitivity during control. Arrow-to-pixels indicate a reduction and circled pixels indicate an increase in sensitivity. Right, the change in sensitivity produced by ultrasound Off. (12e) shows change in sensitivity as a function of distance from the ultrasound focus for On (left) and Off (right) intervals.

FIGS. 13a-13b CdCl₂ abolishes neurostimulation by ultrasound. (13a) shows raster plots of three cells (columns) that responded well to visual and ultrasound stimuli in the control condition. Top and middle rows, Visual and ultrasound responses in the normal Ringer's solution. Bottom, ultrasound response during 100 μm CdCl₂, and Ca²⁺ replaced with Mg²⁺ (13b) shows PSTHs of these three cells, according to embodiments of the invention.

FIGS. 14a-14c. shows how ultrasound acts in part downstream of the photoreceptor to bipolar cell synapse. (14a) left, shows raster plots and PSTHs for visual and ultrasound (20 W/cm²) responses in the control condition for an example cell. Middle, Raster plots and PSTHs after 30 min of 20 μm L-AP4 perfusion. Right, shows raster plots and PSTHs after 60 min of washout. (14b) left, shows istogram of On suppression index for visual stimuli: n=33 (−1, complete suppression of On response; 0, no effect; +1, an On response appears with drug that was not present during control). Right shows histogram of On suppression index for ultrasound stimuli; n=63. The mean was not significantly different from zero (p=0.85, t test). (14c) left, shows visual versus ultrasound suppression indices; n=29 (different distributions: Wilcoxon Signed Rank, p=1.2×10⁻⁷, two-tailed). Diagonal line indicates equal suppression. Right, On-Off index from visual control (1, pure ON cell; −1, pure OFF cell) versus ultrasound suppression index. Black line indicates a linear fit, according to embodiments of the invention.

### DETAILED DESCRIPTION

According to the current invention, a retinal stimulation and prosthetic device is provided that includes at least one ultrasonic transducer having a focused ultrasonic signal, where the focused ultrasonic signal includes an acoustic frequency, a spot size, a temporal pattern, a pulse duration and a power capable of stimulating retinal neurons when the at least one ultrasonic transducer is disposed proximal to an eye.

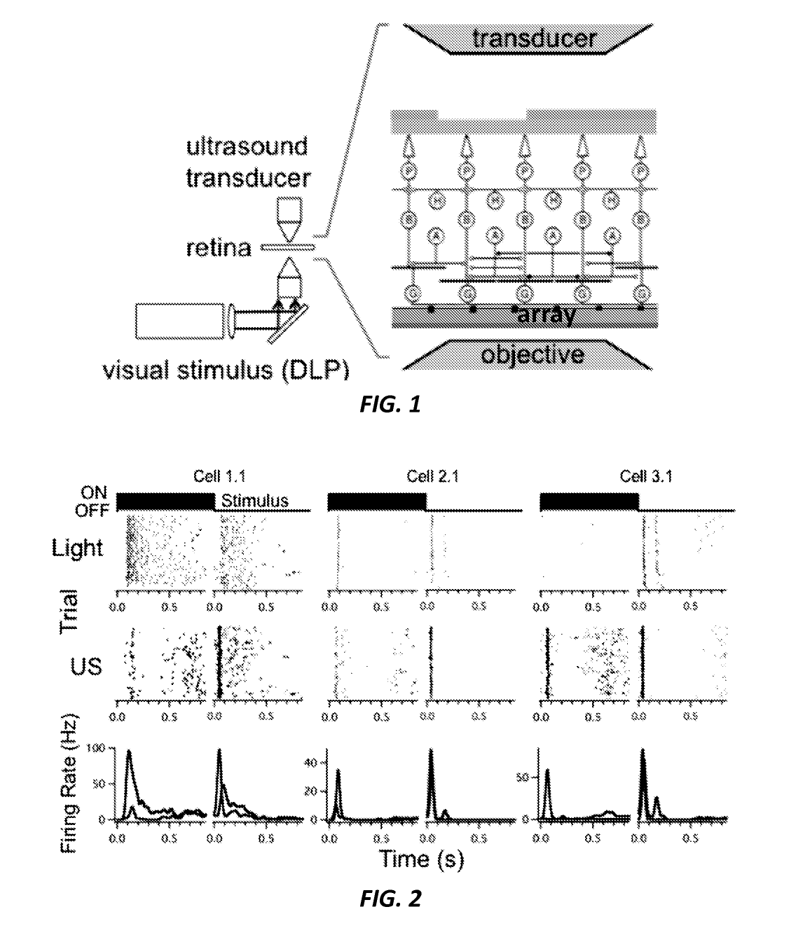

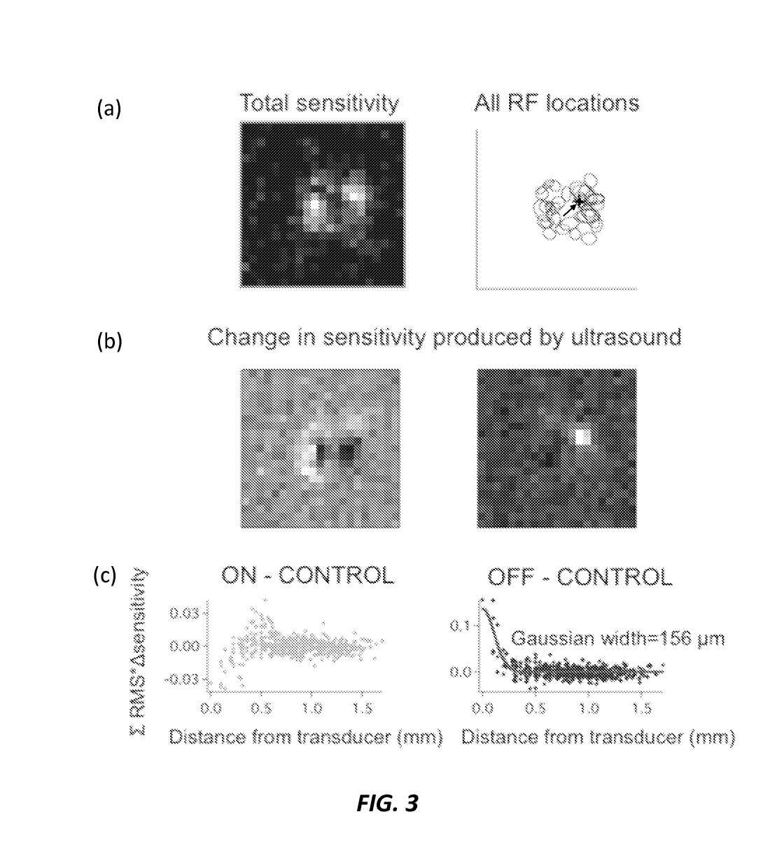

In one example of the current invention, a set of experiments were conducted on an isolated salamander retina that was placed on a multi-electrode array to record spiking output from ganglion cells over an area of ˜1 mm², where FIG. 1 shows a schematic of the experimental setup, according to one embodiment of the invention. The retina was stimulated using both its natural stimulus, light, and ultrasound energy. Visual stimuli were applied by projecting images from a DLP projector focused on the retina. To apply ultrasound stimuli, a custom focused ultrasound transducer with a ˜40-MHz center frequency was used. This transducer with a piezoelectric crystal as an active component and a quartz focusing lens provides a working distance of ˜4 mm, a lateral resolution of ˜100 μm, and a focal depth that spans the full thickness of retina. To excite the transducer a 40-MHz sinusoidal carrier signal was modulated with a 500-kHz square wave with 50% duty cycle, which was turned on and off at an even lower frequency (0.5-15 Hz) with different temporal patterns. The same temporal pattern was also used to turn on and off the visual stimulus. The most important findings in these experiments pertinent to this invention include ultrasound stimulation produces precise, stable responses qualitatively similar to visual responses, where FIG. 2 shows responses of retinal ganglion cells recorded with an array of extracellular electrodes to stimulus onset (ON) and offset (OFF) for a uniform field 0.5 Hz visual flash (data shown in black) and 0.5 Hz ultrasound (data shown in red) stimuli. Further, the important findings include using high-frequency focused ultrasound a high spatial resolution is achieved in retinal neurostimulation, where FIGS. 3a-3c show RMS amplitude of spatio-temporal receptive fields (locations at right), summed over all ganglion cells in a single experiment, where the cross indicates the ultrasound focus (FIG. 3a). Further shown in FIG. 3b is the change in the sensitivity produced by ultrasound stimulus ON (Left) and OFF (Right). FIG. 3c shows the sensitivity as a function of distance from the ultrasound focus during stimulus ON (left) and OFF (right), where the ON gain change shows a center/surround antagonism. In this example, the resolution was at least as precise as 156 μm, however expected resolution from transducer frequency is ˜100 μm. An additional important aspect includes the ultrasound stimulation changes visual sensitivity.

Some exemplary embodiments of the invention for diagnosis and treatment of eye diseases are provided that include, in cases of photoreceptor degeneration, a focused ultrasound transducer can be used to probe remaining retinal function. It was previously not possible to test whether retinal neurons downstream of degenerated photoreceptors are still functional, where the ability to do so would allow physicians to track the progression of complex diseases that involve both photoreceptors and inner retinal neurons. By using ultrasound according to one embodiment of the invention, a patient could report that regions of the retina that are not responsive to light are responsive to ultrasound, indicating that inner retinal neurons are to some extent functional, where ophthalmologists who already use ultrasound as a diagnostic device to image the structure of the eye benefit from the current invention.

According to a further embodiment, in cases where a patient is a candidate for an electronic retinal prosthesis, it is critical that the location of implantation is in the region of a functional inner retina. Ultrasound can be used to identify these regions. Here, the ultrasound beam is scanned over the entire retina, and feedback in this case can be acquired from the patient instead of an electrical readout. Thus, a map can be created of the retina showing remaining neurally functional regions in order to guide the implantation of a retinal prosthesis.

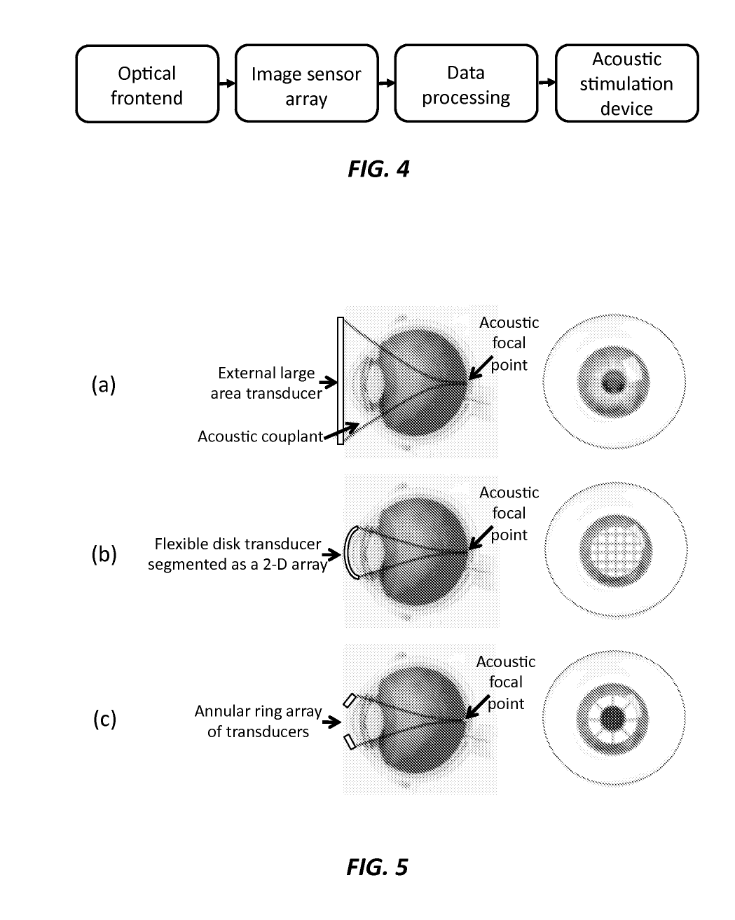

In another embodiment of the invention, a non-invasive prosthetic device for the blind can be implemented in the form of a goggle or a contact lens using the described ultrasound-based neurostimulation technique. In one example, such a system includes a visual image capture device, a data processing unit, and an acoustic transducer or array of transducers, as shown in the flow diagram of FIG. 4. The visual image capture device includes an optical focusing system and an image sensor array and can be conveniently implemented using a standard digital camera. The captured image is then sent to a data processing unit, which can be implemented using a standard microprocessor. A high-resolution acoustic beam is then formed by the acoustic transducer and rapidly scanned over the entire retina in a similar fashion to an electron beam that scans a fluorescent screen in a cathode ray tube, or even more closely similar to a virtual retinal display. Depending on the particular embodiment, the scanning can be achieved mechanically or electronically. This unit uses the pixel brightness information of the acquired image to modulate the intensity of the acoustic excitation during neurostimulation of the retina.

In another embodiment of the invention, provided is a device directed to the excitation of the transducer array by applying signals to reproduce the full scene as visible to the CCD camera thus producing an ultrasound field distribution that reproduces the visible field of view. A Fourier Transform of the field is used to excite the transducers. In this way, one excitation can present the field of view that is equivalent to electronic scanning.

According to another embodiment, an additional element of the system includes data processing of the signal before the visual image is transformed into ultrasonic input. Because the system is designed to replace a damaged part of the visual system, those functions that are lost should be replicated in software. These include spatial and temporal filtering, e.g. edge enhancement, that is produced by the outer retina.

In a further embodiment of the invention, one advantage of the ultrasound excitation approach at a frequency of 50 MHz, for example, is that the focal spot is about 30 microns, which then results in having a number of pixels of excitation over the retina with 30 microns periodicity. Note that the smallest pixel size used in the exemplary implants discussed earlier is 60 microns; this gives the ultrasound excitation a factor of 4 increase in the number of pixels. Increasing the frequency of operation (limited only by attenuation in the liquid inside the eyeball) will translate directly into increased number of excitation pixels.

Further exemplary embodiments of the described device are shown in FIGS. 5a-5c, where FIG. 5a shows a first exemplary device in the form of a goggle where an external acoustic transducer is coupled to the eye using a liquid coupling medium such as water. In this embodiment the acoustic transducer can be a single focused transducer that is scanned mechanically. Alternatively, an array of acoustic transducers can be used for electronic scanning. The acoustic beam intensity corresponding to a bright pixel in the image will be higher. Compared to a contact-lens-like device, this approach allows the use of a larger aperture transducer practically at the same distance from the retina as a contact lens. Hence a smaller acoustic focal spot can be achieved for a given operating frequency. In this embodiment, the image sensor, the data processing unit, the acoustic transducers, and a battery can all be placed on the goggle to implement a standalone system. Because the current embodiment has reflections between the water and the eye, and because the beam from off axis excitation can interact with the skull, this embodiment is not intended to be a permanent prosthetic. More importantly, the device is used as an evaluation tool before implementation of a more elegant solution as described below.

Another embodiment is shown in FIG. 5b that includes a thin conformable disc segmented as a 2-D array of individually accessible transducers to allow electronic scanning. In this embodiment the image sensor, the data processing unit, and the power source are disposed externally and the data and power links between the external unit and the acoustic transducer array can either be achieved through a thin flexible circuit or wirelessly. The disc can cover as much of the front of the eye as feasible, and the transducer can be made of flexible piezoelectric material such as PVDF, or from capacitive micromachined ultrasonic transducers (CMUT), or any other type of transducer that can be made flexible to fit over the eyeball. One exemplary method for making the 2D array is to have one set of parallel lines on one face of the transducer, and another set of lines that is perpendicular to the first set and is placed on the other face of the transducer. In this fashion, a transducer element exists at each intersection of two lines. Access to the metal lines can be provided by a flexible printed circuit board (PCB) that is then connected to excitation and control electronics.

Another embodiment of the invention is shown in FIG. 5c that is in the form of an annular ring array of individually accessible transducers, and includes similar elements to the device shown in FIG. 5b. The central optically transparent opening allows projection of an optical image on the retina. In this case ultrasound stimulation can play an enhancing role.

In contrast to current prosthetic devices used for restoring retinal function are implanted photodiode arrays, the current invention has one or more of the following advantages over existing technology that include noninvasiveness, where no surgery is required, it enables stimulation of the retina with greater spatial resolution, where typically the large diode area required for generating sufficient output current limits the resolution of the photodiode arrays, and using acoustic stimulation at very high frequencies single cell resolution can be potentially achieved.

Some variations of the current invention include the visual image capture device, processing unit, and the acoustic transducer array all can benefit from the continuous scaling of electronic devices and can possibly be all contained in a stack of integrated circuits in the future. Another variation can include by using an array of acoustic transducers multiple beams can be formed simultaneously. In a further variation, the invention is not specific to a particular transducer technology, where conventional piezoelectric transducers or silicon-based micromachined transducers can be used. Micromachined transducers lend themselves to array applications because of advantages such as microlithography-based shape definition and easy integration with electronic circuits. Piezoelectric transducers can be used with no DC biasing or precharging. Capacitive micromachined ultrasonic transducers are used either in constant-voltage or constant-charge operation modes. Further, a virtual retinal display device can be implemented based on the described technology.

An experimental example is provided that uses an isolated retina to characterize the effects of ultrasound on an intact neural circuit, where an isolated salamander retina is used to record the spiking responses of ganglion cells to ultrasound and light using an array of 60 electrodes. A key advantage of the retina is that it can also be stimulated by its natural stimulus, light. Here, ultrasound stimuli near a frequency of 40 MHz were delivered from a piezoelectric transducer in saline at a working distance of 4 mm. Pulse trains lasting 3-30 microseconds continued for one second, and were presented at a frequency of 0.5 Hz. The focal spot was 50 microns in diameter and spanned the retina in depth. For comparison of ultrasound responses to light responses, also presented is a flashing light at 0.5 Hz.

Strong ultrasound stimuli evoked precise responses that looked qualitatively similar to strong visual responses. Ultrasound responses were stable for 300 s, contained ON and OFF transients of different types, and showed sustained activity. Temporal jitter at stimulus offset was comparable between light and ultrasound stimuli, and was often less than 10 ms. However, the fastest ultrasonic latencies were shorter than the fastest visual latencies. Further, the relative strength of OFF vs. ON response for the ultrasound stimulus was often very different from those of the flash, as were the response kinetics. This indicates that ultrasound stimuli activated some cells downstream of photoreceptors. The effects decayed to half maximal over 300 μm, considerably larger than the ultrasound stimulus focal spot. This lateral spread is within the spatial scale of lateral connections, including those from horizontal and amacrine cells. Ultrasound is thus likely stimulating interneurons within the circuit.

These results indicate that ultrasound stimulation is an effective and temporally precise method to activate the retina downstream of photoreceptors. Because the retina is the most accessible part of the central nervous system in vivo, ultrasonic stimulation may have diagnostic potential to probe remaining retinal function in cases of photoreceptor degeneration, and therapeutic potential for use in an electronic retinal prosthesis. In addition, ultrasound provided by the current invention provides for a basic understanding of dynamic activity in the interneuron population of the retina. More specifically, in this example ultrasound stimuli at an acoustic frequency of 43 MHz and a focal spot diameter of 90 μm delivered from a piezoelectric transducer evoked stable responses with a temporal precision equal to strong visual responses but with shorter latency. By presenting ultrasound and visual stimulation together, it was found that ultrasonic stimulation rapidly modulated visual sensitivity but did not change visual temporal filtering. By combining pharmacology with ultrasound stimulation, it was also found that ultrasound did not directly activate retinal ganglion cells but did in part activate interneurons beyond photoreceptors. These results show that, under conditions of strong localized stimulation, timing variability is largely influenced by cells beyond photoreceptors.

The invention provides ultrasonic stimulation as an effective and spatiotemporally precise method to activate the retina. Because the retina is the most accessible part of the CNS in vivo, the ultrasonic stimulation of the current invention provides diagnostic potential to probe remaining retinal function in cases of photoreceptor degeneration, and therapeutic potential for use in a retinal prosthesis. In addition, because of its noninvasive properties and spatiotemporal resolution, ultrasound neurostimulation provides a useful tool to understand dynamic activity in pharmacologically defined neural pathways in the retina.

In the current exemplary experiment, multielectrode recordings were performed. The isolated retina of a tiger salamander of either gender was adhered by surface tension to a dialysis membrane (˜100 μm thick) attached to a plastic holder. It was then placed on a motorized manipulator and lowered onto a 60-electrode array (ThinMEA, Multichannel Systems) ganglion cell side down. A low-density array (8×8 grid, 100 μm spacing) was used with uniform field and checkerboard visual stimuli, and a high-density array (two 5×6 grids with 30 μm electrode spacing, the grids separated by 500 μm) when using a 100 μam spot visual stimulus centered over one grid.

The ultrasonic transducer was a custom-made, focused delay line transducer with a Lithium Niobate active element and a fused quartz focusing lens, and was operated at the designed center acoustic frequency of 43 MHz.

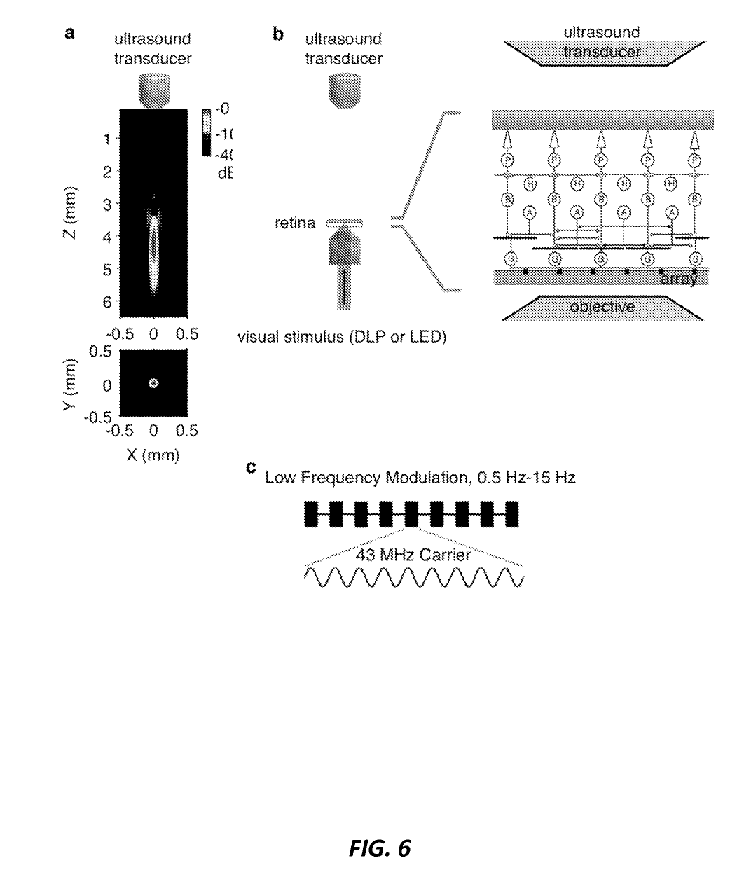

The acoustic frequency was chosen to yield a focal spot smaller than the receptive field center of a ganglion cell but was not varied for this initial study. It was mounted on a micromanipulator (model MPC-385-2, Sutter Instruments) and immersed in the perfusion fluid above the retina as shown in FIG. 6b. Ultrasound propagated from the transducer, through the water bath, dialysis membrane, retina, and reflected off the multielectrode array. Some energy was also reflected off the dialysis membrane and retina interfaces, and this could be used in ultrasound imaging mode to determine the proper depth of the transducer for retinal stimulation. A function generator (model 8116A, Hewlett-Packard) was used to produce the 43 MHz carrier, which was gated on and off by the analog output from a National Instruments data acquisition board and then passed through a 50 dB RF power amplifier (model 320 L, Electronic Navigation Industries) to stimulate the custom transducer. The focal length of the transducer was 4.3 mm, with a lateral resolution estimated to be ˜90 μm, and a focal zone that spans the retina in-depth (see FIG. 6a for a simulation of the spatial power distribution). It is difficult to measure the power output of a 43 MHz transducer because typical hydrophones are not calibrated for that frequency and do not have sufficient spatial resolution. Therefore, the insertion loss from 20 to 50 MHz is measured. Power was measured at 20 MHz using a laser interferometer (model OFV-511, Polytec), and the expected power density at 43 MHz was calculated using the insertion loss curve. The calculated time-averaged acoustic power was 10-30 W/cm² for 50% duty cycle stimulus (e.g., 1 s On, 1 s Off) for most experiments.

The 43 MHz carrier was modulated at low frequencies (0.5-15 Hz) to match the temporal pattern used for visual stimulation (FIG. 6c). For most experiments, this included 1 s of stimulus On and 1 s of stimulus Off, repeated for many cycles, for a total duration of 1-5 min. In some experiments, the On and Off times were varied randomly to make a binary noise stimulus sampled at 30 Hz to match the temporal structure of visual stimuli presented from a video monitor.

To position the ultrasound transducer, the reflected signal from the MEA was detected by the transducer in imaging mode. To adjust the tilt angle so that it was orthogonal to the MEA and to position the focal point at the depth of the retina, the reflected signal was maximized. To calibrate the lateral position of the ultrasound transducer relative to the MEA, a small pinhole (˜200 μm) in a piece of aluminum foil was positioned over the center of the array, as confirmed by a CCD camera image. Next, the reflected signal from the edge of the hole was used to determine the lateral boundaries of the pinhole edge. Then the transducer was moved laterally so that the focus was centered over the hole. For the low-density array, the calibrated transducer position was in the center of the array. For the high-density array, the transducer was positioned in the center of one of the two groups of electrodes.

In early experiments, visual stimuli were uniform field flashes from a red LED. To generate spatial stimuli, later experiments used a DLP projector (model 2300 MP, DELL) focused on the retina from below. The output of the projector was attenuated by neutral density filters and adjusted so that the photopic mean intensity was ˜10 mW/m². Visual stimuli had the same temporal pattern (1 s On, 1 s Off, or binary random noise) as used for ultrasound stimulation to facilitate a direct comparison. To measure spatiotemporal visual sensitivity in the presence or absence of ultrasound stimuli, a spatial checkerboard with random, binary modulation of 100 μm squares was used.

Spatial receptive fields and temporal filters were calculated by the standard method of reverse correlation with the spatial checkerboard visual stimulus consisting of binary squares, such that:

$$F ( x , y , τ ) = {\int }_{0}^{T} s ( x , y , t - τ ) r ( t ) d t , \qquad ( 1 )$$

where F(x, y, τ) is the linear response filter at position (x, y) and delay τ, s (x, y, t) is the stimulus intensity at position (x, y) and time t, normalized to zero mean, r(t) is the firing rate of a cell, and T is the duration of the recording. The filter F(x, y, τ) was computed by correlating the visual stimulus to spike times for ganglion cells. A temporal filter was computed as the spatial average of F( ). For the ultrasound and visual spot binary modulation, F(x, y, τ) becomes F(τ) and s (x, y, t) becomes s(t) as there is no spatial dimension. When computing linear-nonlinear (LN) models, the filters were normalized in amplitude such that the SD of the filter input and output was equal. This placed total sensitivity in the averaged slope of the nonlinearity.

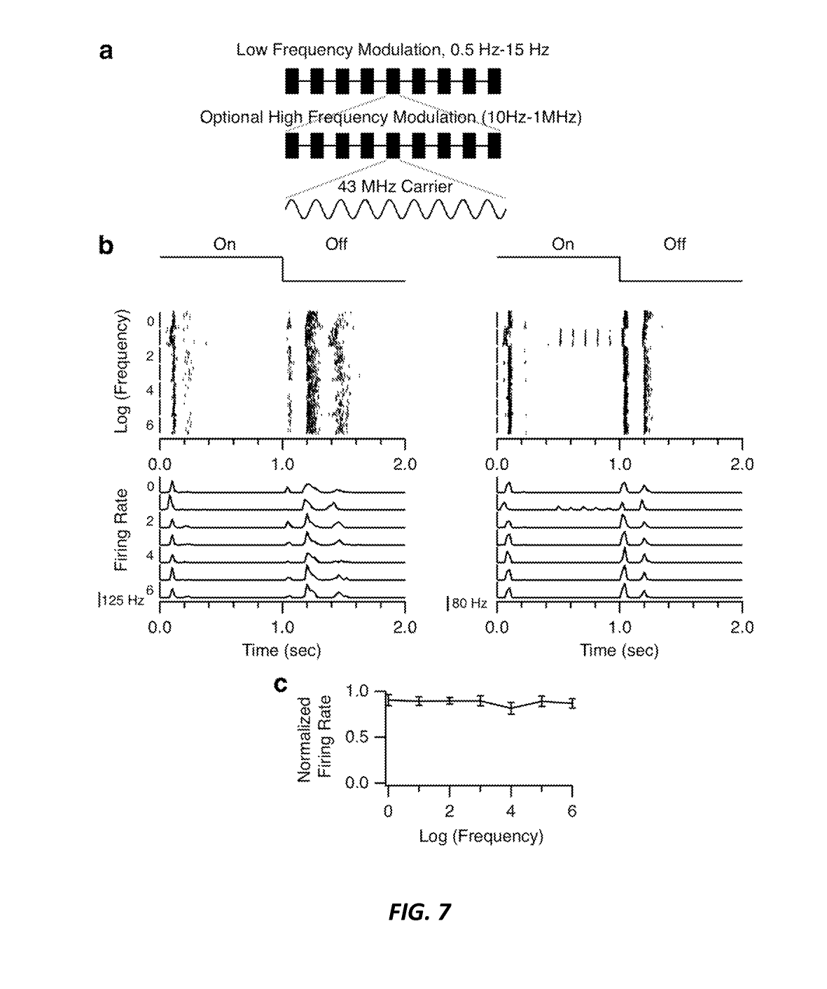

Ultrasound stimuli (43 MHz) repeated at a stimulus frequency of 0.5 Hz generated reproducible activity in retinal ganglion cells (FIGS. 7a-7c). Normal visual responses occur in a frequency range of ˜0-15 Hz. Previous results in hippocampal slices at an acoustic frequency of ˜0.5 MHz suggest that modulating the ultrasound carrier in the kilohertz range increased the efficiency of ultrasound neurostimulation. There exists some resonant frequency or ideal pulse length that optimally stimulates cells. Therefore, in addition to the repetition at physiological frequencies (e.g., 0.5 Hz), it was tested whether a further higher frequency modulation between 0 and 1 MHz affected neural activity (FIG. 7a). Within each 1 s stimulus pulse, the duty cycle (50%) and thus the average power was kept constant, and high-frequency pulse duration varied inversely with modulation frequency. It was found that neither modulation frequency nor pulse duration had any effect on responses when average power was held constant (FIGS. 7b and 7c). Only stimulus frequencies within the physiological range (<15 Hz) affected neural activity. Other experiments changing the duty cycle (data not shown) suggested that only average power is important, neither a high-frequency modulation nor pulse duration matters with a 1 s total duration.

Therefore, in subsequent experiments, the high-frequency modulation was estimated. A continuous waveform is advantageous because it has the lowest peak power for a given average power, reducing any possible negative effects on a cell that depend on the peak stimulus power. Furthermore, the absence of a modulation frequency or pulse duration provides information about the biophysical mechanism transducing the ultrasound stimulus. For ultrasonic stimuli at 43 MHz, the primary mechanisms for ultrasound transduction in the retina do not appear to have any resonance or frequency preference in the range of 15 Hz to 1 MHz.

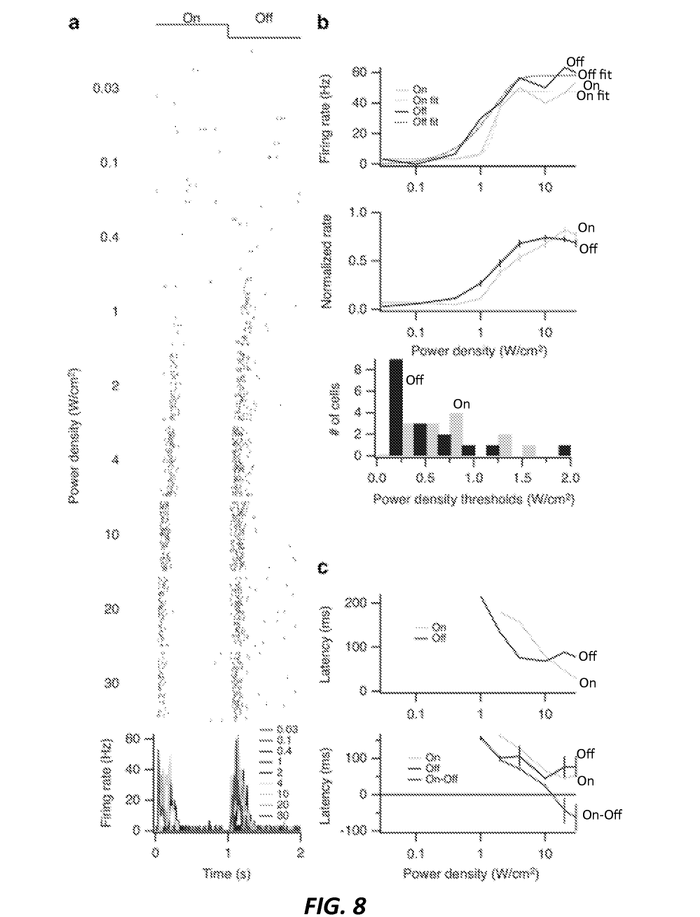

A minimum power level was sought that generated a robust, reproducible response similar to a visual response. A stimulus frequency of 0.5 Hz was used, and average power was varied between 0.03 and 30 W/cm2 (FIG. 8a). Generally, firing rate increased with power until the response saturated. Responses reached a maximum, on average at 10-30 W/cm2 (FIG. 8b). Therefore, a power level is chosen in this range for most experiments. At 30 W/cm², steady heating was measured with a thermocouple at ˜0.5° C. Latency varied greatly with power for some cells, with lower power producing longer latencies (FIG. 8c). In FIGS. 8a-8c shown are the dependence of response on ultrasound power density, where FIG. 8a (top) shows raster plots of a single cell at increasing ultrasound power levels and the (bottom), superimposed PSTHs (10 ms bins), FIG. 8b shows (top) peak firing rates for On and Off responses for one cell versus power density (solid lines) along with sigmoid fits (dotted lines), and (middle) a population summary, On (n=29) and Off (n=32) responses shown separately, where the peak firing rate of each cell was normalized to its maximum rate, and error bars indicate SEM (Bottom), for cells that were fit well by sigmoid, a threshold was defined at 5% of the minimum-maximum range. A histogram of those thresholds is shown (On median=754 mW/cm², Off median=250 mW/cm², Wilcoxon-Mann-Whitney two-sample rank test: p=0.0033, one-tailed). FIG. 8c shows (top), Latencies to first spike for the example cell in FIG. 8a (bottom), population summary of average latencies and error bars indicate SEM, according to one embodiment of the invention.

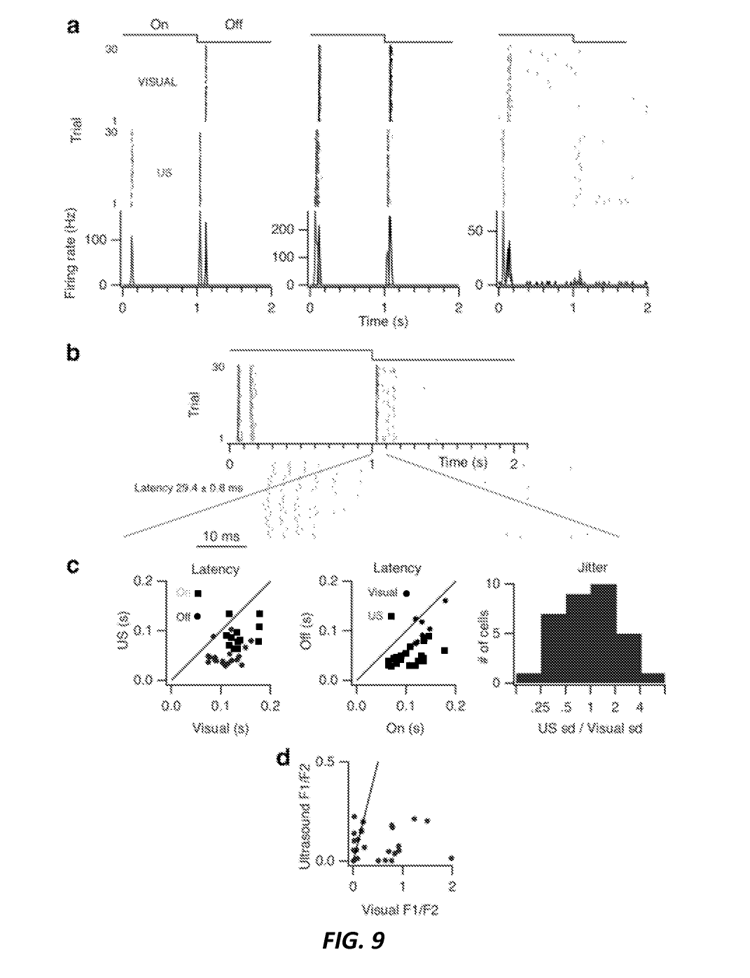

In comparing visual responses to ultrasound responses (simple periodic stimuli), the responses of individual ganglion cells to an ultrasound stimulus (43 MHz) modulated at 0.5 Hz were strong and reproducible, much like visual responses to a 0.5 Hz flashing 100 μm spot that illuminated the same area as the ultrasound stimulus (FIG. 9a), where shown are aster plots (30 trials) and PSTHs of three cells for both visual and ultrasound stimuli, 0.5 Hz, showing Off type (left), On-Off type (middle), and On type (right) ganglion cells

For some cells, the firing rate and duration of the responses were similar, except that latency of the ultrasound response was shorter than visual latencies (FIG. 9a, middle and right). For other cells, ultrasound stimuli generated both ON and OFF responses, whereas visual stimuli generated only OFF responses (FIG. 9a, left).

It was found that ultrasound stimulation produced precisely timed spikes across multiple repetitions (FIG. 9b), where shown (top) are ganglion cell recorded with a multielectrode array responding to a 0.5 Hz ultrasound stimulus. Stimulus trace (middle) showing amplitude of ultrasound stimulus. Raster plot of spiking activity (bottom) from repeated trials for a ganglion cell. Expanded trace beginning at the offset of the ultrasound pulse. Periodic neural activity is consistent with refractoriness. Transient bursts of action potentials occurred both at the onset and offset of the ultrasound pulse. For cells that responded to both visual and ultrasound stimulation, the latency of the ultrasound response was on average considerably shorter than the visual response as shown in FIG. 9c, where (left) visual On, 139±3.3 ms, ultrasound On, 98 ms±4.5 ms, two-tailed paired t test, p=6.5×10⁻⁶, n=14; visual Off, 111±3.8 ms, ultrasound Off 49±2.8 ms, two-tailed paired t test, p=5.9×10⁻⁸, n=19). It is likely that this latency difference arises because the ultrasound stimulus acts later in the circuit, at the very least bypassing the phototransduction cascade. For visual responses, latencies were longer for On than for Off responses, arising because On and Off signals are conveyed by different neural pathways containing On and Off bipolar cells, respectively. In FIG. 9c (left), comparison of ultrasound and visual latencies to first spike for cells in which the peak of the PSTH exceeds 25 Hz (On, correlation coefficient r=0.34, p=0.12; Off, r=0.2, p=0.2). Similarly, ultrasound responses had a longer latency for On than Off responses, suggesting that two types of ultrasound responses also traveled through different neural pathways shown in FIG. 9c (middle). Here, Comparison of On and Off latencies for visual responses (r=0.58, p=0.0024) and for ultrasound responses (r=0.75, p=0.01). Further, in FIG. 9c (right) jitter was computed for each cell for ultrasound and visual responses as the SDs of the first spike latencies. Shown is a histogram of the ratio of ultrasound jitter to visual jitter for each cell (median=0.88, paired t test p=0.904). FIG. 9d shows the ratio of power at the fundamental frequency (F1, 0.5 Hz) to power at the second harmonic (F2, 1.0 Hz) for ultrasound and visual stimuli (r=0.006, p=0.49).

The temporal precision of neural responses was similar across the population between ultrasound and visual stimuli shown in FIG. 9c (right) and in some cases was smaller than 1 ms, as has been reported for visual stimuli (FIG. 9b). Thus, even though the latency was significantly shorter, the jitter was not significantly different in FIG. 9c (right), median=0.88, paired t test, p=0.904). This suggests that, under these visual stimulus conditions, the variability in latency is not substantially influenced by the phototransduction cascade, but by later circuit elements.

To measure the relative strength of On and Off responses, the frequency response of cells was analyzed to the ultrasound stimulus presented at 0.5 Hz. The response was compared at the fundamental (F1=0.5 Hz) frequency and at the second harmonic (F2=1 Hz). Cells that only respond to onset or offset will have a strong F1 component, whereas cells that respond equally strongly to both onset and offset will have a strong F2 but weak fundamental response. In 48% of cells, the ratio of the fundamental to the second harmonic was much less for ultrasound than for visual stimuli as shown in FIG. 9d. This difference between ultrasound and visual responses suggested that ultrasound signals travel to some extent through different neural pathways than visual stimuli, therefore indicating that ultrasound stimuli in part activate cells other than photoreceptors. Further experiments and analyses to address this issue are presented below.

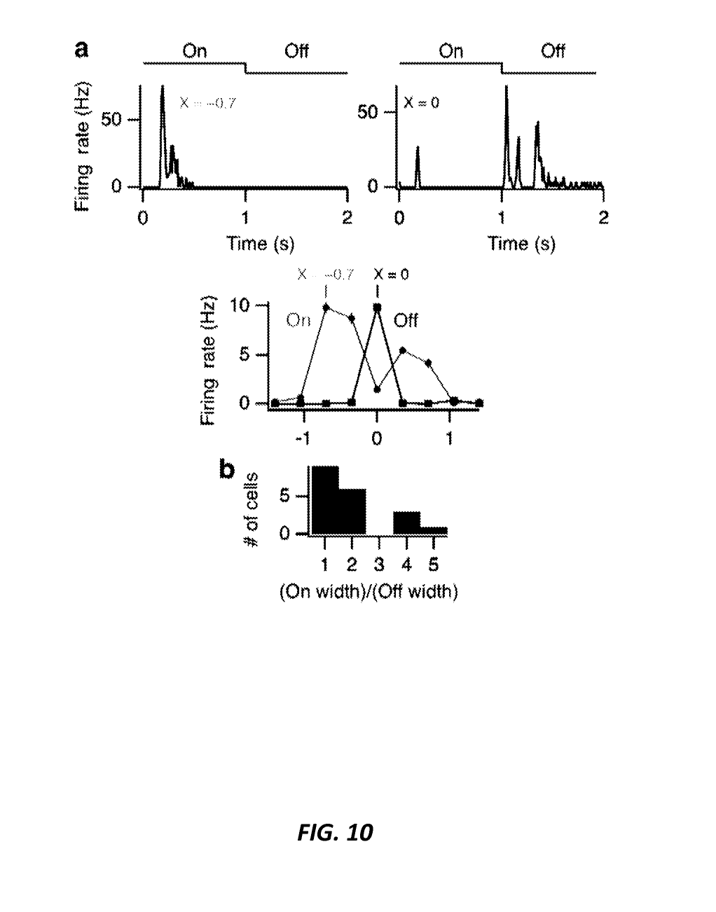

The response to ultrasound stimuli as a function of distance from the ganglion cell was then measured. Retinal ganglion cells have a spatially antagonistic receptive field, with a surrounding area that responds to light with the opposite sign as the receptive field center. To measure whether this spatial antagonism was present in ultrasound responses, the transducer was moved in relatively large steps (350 μm), as the receptive field surround can extend to 1 mm radius. In the example shown in FIG. 10a, the cell responded mostly to ultrasound Off when the stimulus was placed over the receptive field center (FIG. 10a, right and bottom, x_{—}0), but responded to ultrasound On only when the stimulus was moved 700 μm away (FIG. 10a, left and bottom, x=−0.7 mm). As is the case with visual stimuli, the antagonistic surround spanned a larger region than the receptive field center (FIG. 10b). This effect indicates processing within the retinal network, implying that ultrasound stimuli in part stimulated cells other than ganglion cells directly.

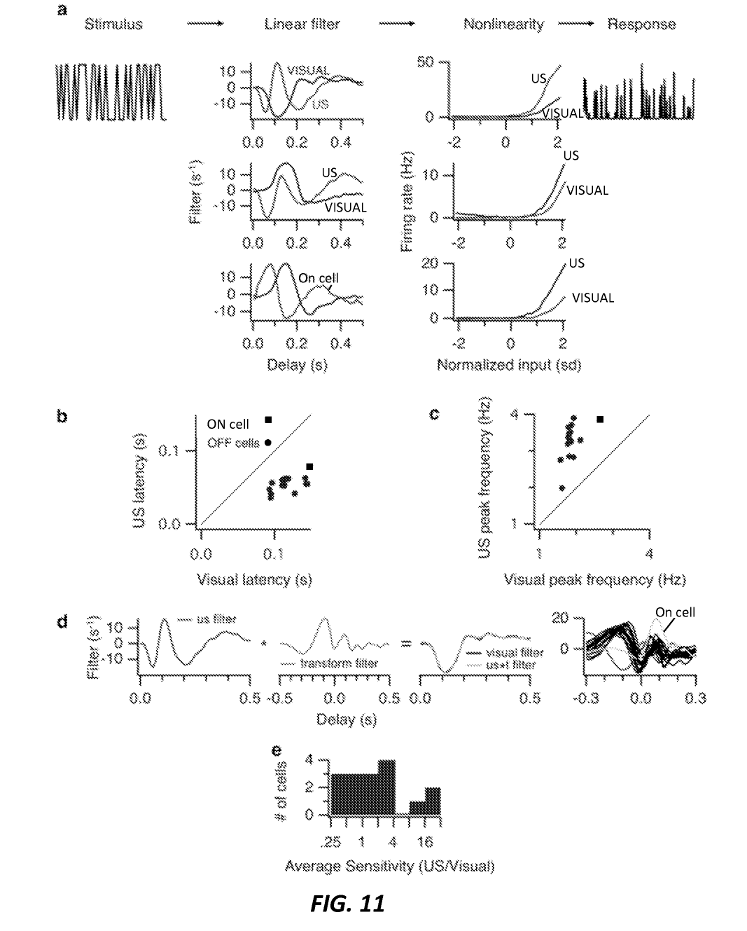

Ganglion cell visual responses can be approximated by a model containing a linear temporal filter followed by a static nonlinearity, where in this LN model, the temporal filter represents the average change in firing rate in response to a brief pulse of light, and the nonlinearity is a time-independent function that captures the sensitivity, threshold, and any saturation in the response. To compute LN models, the ultrasound stimulus was modulated in time with binary noise. This was compared with a visual LN model computed by modulating a 100 μm spot visual stimulus with the same binary noise. The linear filter was calculated by the standard method of reverse correlation as the time-reverse of the average stimulus preceding a spike. After convolving the stimulus through this filter, a static nonlinearity was computed as the average instantaneous relationship between the filter output and firing rate (FIG. 11a). The filters were normalized in amplitude so that the total sensitivity was represented in the average slope of the nonlinearity.

Ultrasound filters (FIG. 11a, left) had a much shorter latency and time to peak compared with visual filters, as expected from the shorter latency of periodic pulses of ultrasound stimuli (FIG. 9). Ultrasound filters were also very strongly biphasic, even triphasic, meaning that they were differentiating or high-pass filters, reflecting transient responses.

Other differences that were seen between ultrasound and visual filters observed occasionally were that the ultrasound filter had the opposite polarity from the visual filter (2 of 17 cells) (FIG. 11a, middle), or that the ultrasound and visual filter had similar dynamics, but a different latency (1 of 17 cells) (FIG. 11a, bottom).

Additional diversity was observed between visual and ultrasound nonlinearities (FIG. 11a, right). In general, the average sensitivity for the ultrasound response could be greater (7 of 16 cells) (FIG. 11a, top), less than (3 of 16 cells) (FIG. 11a, bottom), or approximately equal to (within a factor of 2, 6 of 16 cells) the sensitivity of the visual response. The average sensitivity for ultrasound and visual nonlinearities is compared in FIG. 11e.

The hypothesis that ultrasound stimulated photoreceptors only was then considered and that the only difference from visual stimulation comes from bypassing the phototransduction cascade. If this were true, then the differences in the visual and ultrasound filters could be explained by another fixed linear, causal filter that did not vary from cell to cell. For that purpose, filter characteristics across the population are summarized in FIGS. 11b-d, looking at the time to first peak (FIG. 11b) and the peak stimulus frequency measured from the Fourier transform of the filter (FIG. 11c). For Off cells, visual latencies were more diverse than ultrasound latencies; there was not a single number to describe the difference.

Then for each cell, we explicitly computed the filter that would transform the ultrasound filter into the visual filter. This represented the temporal filtering bypassed by the ultrasound stimulus (FIG. 11d). It was found that this transforming filter between ultrasound and visual stimuli varied across cells (FIG. 11d, right). Furthermore, it included a substantial acausal component (to the left of zero in FIG. 11d, far right), which was inconsistent with a single initial filtering step that was bypassed by the ultrasound stimulus. The average normalized root mean squared (RMS) difference between the transformed ultrasound filter and the visual filter was 19.9±8.0% using an acausal filter. When the filter was constrained to be causal by setting it to be zero in the acausal direction, this RMS difference increases to 87.7±13.8%, indicating that causal filter was insufficient. Thus, it is unlikely that ultrasound stimulated photoreceptors alone.

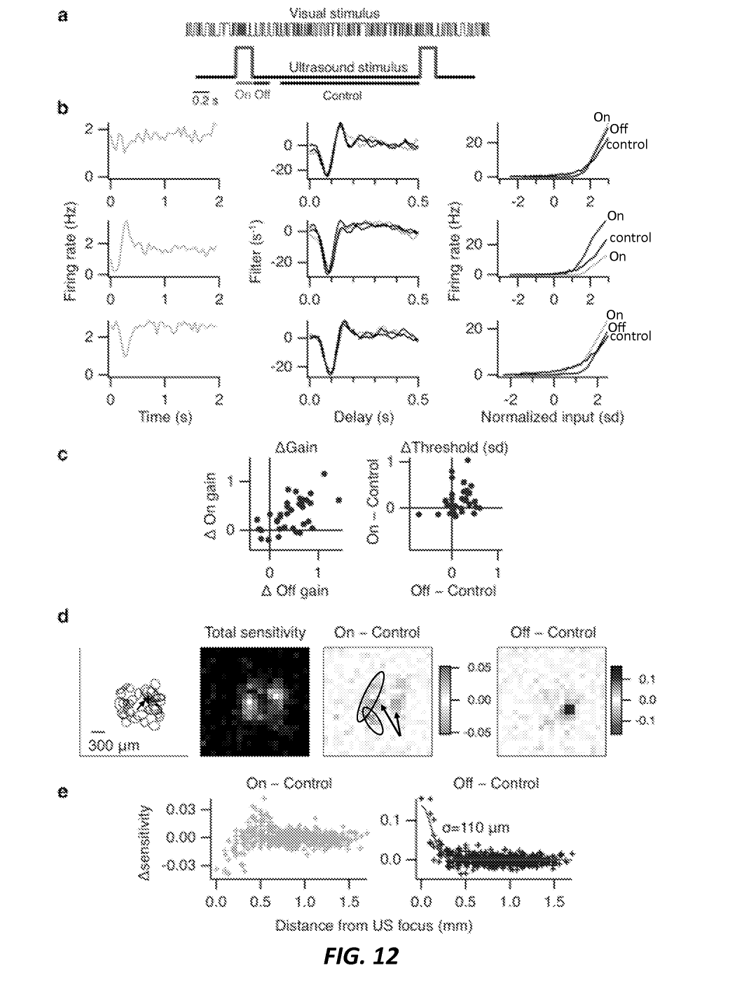

By applying both visual and ultrasound stimuli simultaneously, measured was how ultrasound modulates the normal processing of visual input. A visual stimulus composed of a binary random checkerboard was used, from which the linear spatiotemporal filter, a single static nonlinearity, and the two dimensional spatial receptive field were computed. During this visual stimulation a periodic ultrasound pulse of 200 ms duration was delivered every 2 s (FIG. 12a). The data was analyzed by correlating response to the visual stimulus, and was subdivided into three time intervals, 1) the 200 ms during which the ultrasound pulse was turned on (‘On’), 2) the 200 ms immediately after the ultrasound pulse was turned off (‘Off’), 3) a control period that extended from 300 ms after the pulse was turned off until the next pulse (‘Control’). The Off and Control periods were defined by first analyzing the response in multiple 200 ms intervals, and determining that these three time intervals were representative of the dynamic changes.

At ultrasound onset or offset, many cells briefly changed their firing rate (FIG. 12b). However, during these changes in firing rate, there was virtually no change in the visual temporal filters, except in some cases noise increased because of a lower firing rate. Visual nonlinearities changed in accordance with the change in firing rate. FIG. 12c compares changes in the threshold (lateral shift) and average sensitivity (vertical scaling) of the nonlinearity at ultrasound On and Off (FIG. 12c). Most cells (74%) showed increases in sensitivity both during the On and Off periods relative to the Control period, changes that were weakly correlated in the two time intervals (FIG. 12c, left, correlation coefficient r=0.57, p=0.00017). For less than half of the cells (43%, FIG. 12c, right), threshold increased for both On and Off periods of ultrasound. Considerable diversity was observed in these changes in sensitivity and threshold. In summary, the ultrasound stimulus generally did not fundamentally change temporal filtering but did change threshold and sensitivity in a manner that greatly differed between cells.

Because the spatial visual stimuli enable a very localized measurement of visual sensitivity across a population of ganglion cells, we used the visual spatial receptive field maps to derive an upper limit on the spatial scale of the ultrasound stimulus. FIG. 12d (far left) shows the spatial distribution of visual receptive fields relative to the ultrasound transducer location (arrow directed to “+”). A spatial map of total visual sensitivity across the population of cells was first computed by summing the RMS amplitude of each cell's spatiotemporal receptive field for each spatial location (FIG. 12d, left middle). Then, for each cell, the slope of the nonlinearity was calculated as an estimate of the sensitivity for the three conditions (On, Off, Control). The amplitude of each receptive field was weighted by the change in sensitivity created by ultrasound On or Off conditions compared with Control, and these results were summed across all cells. This yielded a spatial map of the total change in sensitivity produced by ultrasound. A Gaussian fit during the Off interval has an SD of 110 μm. At the onset of ultrasound stimulation, regions near the transducer showed a reduction in sensitivity, whereas regions far away that experience an increase in sensitivity (FIG. 12d, middle right). For these distant cells, ultrasound likely stimulated the receptive field surround. At the offset of the ultrasound stimulus, we observed a spatially localized increase in sensitivity (FIG. 12d, far right). A summary of the average change in sensitivity as a function of distance from the ultrasound focus is shown in FIG. 12e. A Gaussian fit to the effect at the offset of the ultrasound stimulus shows an standard deviation of 110 μm, which can be considered an upper limit on the spatial resolution of the ultrasound transducer. This measure of resolution is affected by the lateral spread of the signal inherent in retinal circuitry, so the actual spatial scale of stimulation may be smaller.

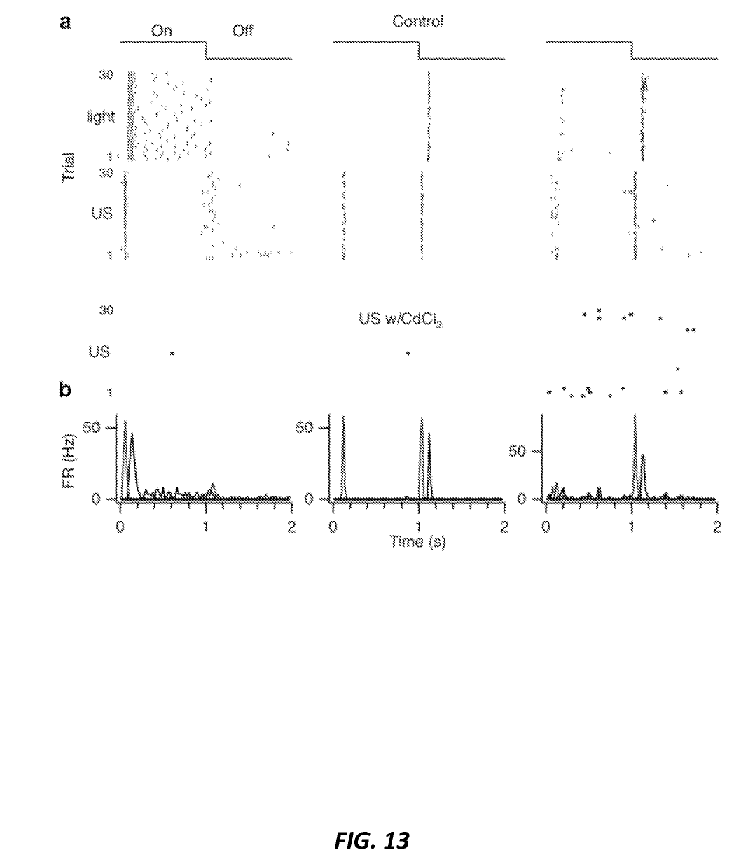

The previous results imply that ultrasound stimuli are processed in retinal circuitry and that ganglion cells are not exclusively stimulated directly. To directly measure the effect of ultrasound on ganglion cells, vesicular transmitter release was blocked. This was accomplished by perfusing the retina with 100 μm CdCl₂, and replacing Ca with Mg. This yielded a higher than normal level of spontaneous activity, which was potentially useful in the detection of any decreases in activity. Ultrasound stimulation at a stimulus frequency of 0.5 Hz was applied for 60 s. Before perfusing CdCl₂, responses to the 100 μm visual spot and ultrasound stimuli at the normal power level (30 W/cm²) were measured as a control to verify normal stimulation (FIG. 13a).

While perfusing CdCl₂, ultrasound stimulation (30 W/cm²) produced virtually no response (FIG. 13b). The stimulus was repeated at progressively higher-power levels up to 180 W/cm², but at no point did we obtain any stimulus-locked response; at most, there was some slow modulation of spontaneous activity (data not shown).

The sum of the fundamental and second harmonic of the response for 19 cells were computed and it was found that none of these cells responded to ultrasound stimuli in the presence of CdCl₂ (response was 2.8±1.5% of control). Thus, ultrasound neurostimulation does not appear to directly activate ganglion cells, and requires synaptic transmission. One possibility for this effect is that ultrasound stimulation of the retina either results in small membrane potential changes that require amplification by synapses before ganglion cells, or that the effect may be directly on synaptic release. In either case, the effect does not appear to be a general effect on the membrane or on all voltage-dependent ion channels.

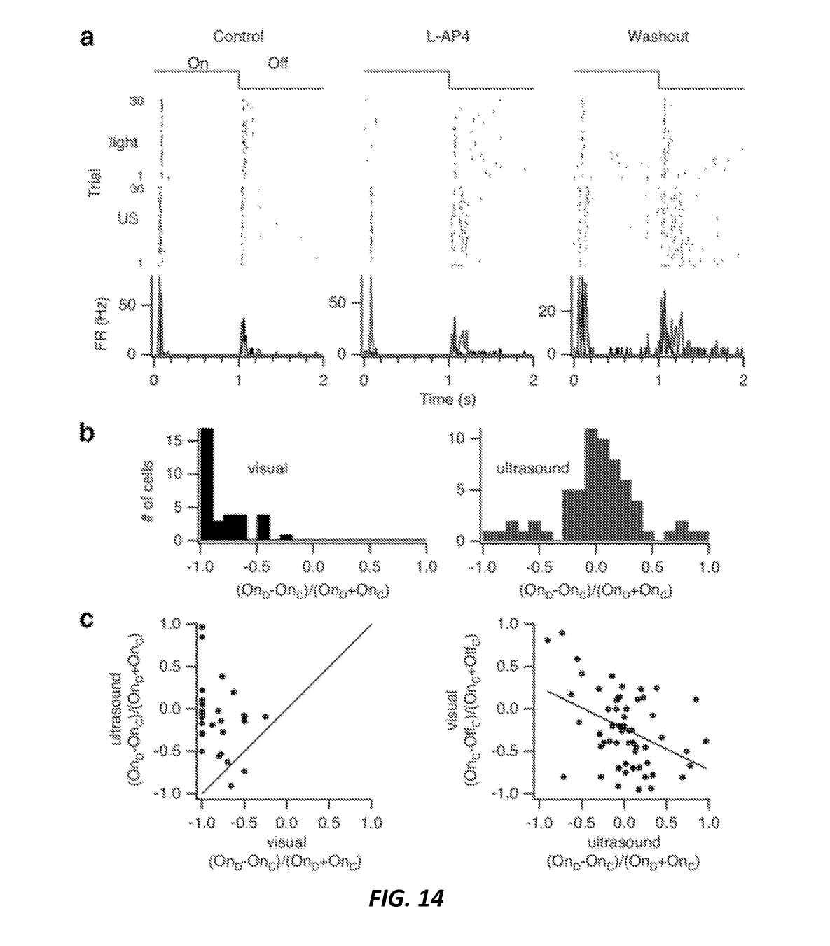

Further tested was whether ultrasound acted on photoreceptors alone by blocking synaptic transmission in the On pathway with L-AP4. Because L-AP4 acts selectively on the synaptic input to On bipolar cells, if ultrasound acted solely through photoreceptors, L-AP4 should also block ultrasound stimulation through the On pathway. The responses to both ultrasound and visual stimulation were measured in the presence and absence of L-AP4 and it was found that all visual responses at the onset of light were suppressed by L-AP4 as expected. However, the response to the onset of ultrasound was in general unaffected by L-AP4 (FIGS. 14a-14c). When analyzing results across the population, however, a weak but significant correlation between the strength of the On response in the cell and the strength of the effect of APB was observed (FIG. 14c, far right). Approximately 18% of the variance of the effect of APB could be accounted for by a difference in the strength of the On response (r²=0.18, p=0.0013, two tailed). This indicates that, to a weaker extent, ultrasound also stimulates photoreceptors. Overall, it is conclude that, because L-AP4 largely did not block the On response to ultrasound, a substantial part of the direct effect of ultrasound stimulation is on cells beyond photoreceptors.

It was shown that ultrasound stimulation can be used to convey precise temporal information across a range of signals similar to natural visual input. The use of a high acoustic frequency further enables a fine lateral spatial resolution (˜100 μm), consistent with the maximum resolution of the 43 MHz frequency (FIG. 13). Furthermore, ultrasound stimulation both indirectly activates ganglion cells independent of visual stimulation and rapidly modulates sensitivity to natural visual input. With regard to the optimal stimulus parameters, high-frequency modulation is found to be unnecessary, neither beneficial nor detrimental. Low-frequency modulation was effective in the normal physiologic range equivalent to the natural visual stimulus.

For clinical use as a prosthesis, it is critical to deliver sensory information at a spatial and temporal resolution and range similar to that of natural visual input. Furthermore, this information must be delivered to existing neurons in the degenerated retina. Similarities between ultrasound and visual responses, and a center/surround receptive field structure measured with ultrasound (FIG. 10), all indicate that retinal circuitry processes the ultrasound signal. Furthermore, although ultrasound did not directly stimulate ganglion cells (FIG. 13) when synaptic transmission was blocked, it did activate cells beyond photoreceptors (FIG. 14). Finally, because many patients with retinal disease may have some existing natural vision, it is important to understand how ultrasound stimuli modulate visual sensitivity. Although ultrasound modulates visual sensitivity (FIG. 12), these effects are highly localized. The results indicate that ultrasonic neurostimulation of the retina may be useful in a clinical setting for diagnosis of retinal health in the absence of intact photoreceptors, and potentially as a noninvasive retinal prosthesis.

For basic studies of neural circuits, although each artificial stimulus method has limitations in spatiotemporal resolution or cellular specificity, these might be overcome by combinations with other methods. In particular, extracellular methods of stimulation often lack specificity in terms of cell types. By combining ultrasound stimuli with specific pharmacology, as we have done (FIGS. 13 and 14), one could potentially understand the effects of pharmacologically defined neural pathways with the spatiotemporal specificity of the ultrasound stimulus.

This combination of ultrasound and pharmacology has revealed new information about the potential biophysical mechanism of ultrasound stimuli. Because ultrasound stimuli do not directly change the firing rate of ganglion cells (FIG. 13), this argues against a nonspecific effect on all cells, such as a transient disruption of the cellular membrane. Another potential mechanism is an effect on voltage-dependent or mechanosensitive ion channels, but only if these effects are specific to different cell types, either resulting from their set of ion channels or their cellular geometry. A final possibility is a direct effect on the presynaptic terminal. If so, this effect must be felt at or before the step of Ca influx. If the effect were direct upon the machinery of vesicle fusion subsequent to the effect of Ca²⁺, it would likely not be sensitive to Cd⁺.

Given that ultrasound acts in part on cells beyond photoreceptors, this knowledge can be used to interpret the origin of certain aspects of neural signaling, one example being the source of variability in retinal processing.

It is known that, for strong visual stimuli, the temporal precision of ganglion cells can exceed 1 ms. However, the retinal elements that establish this limit on temporal precision are unknown. It is thought that the major source of noise in the retina comes from photoreceptors, although these conclusions come from analyzing the statistics of single photoreceptors. It was found, however, that although ultrasound responses have a much shorter latency, they do not have less variability under the stimulus conditions tested (FIG. 9). Thus, under the stimulus conditions of a strong flashing spot, noise in photoreceptors does not seem to have the dominant influence over ganglion cell variability. One explanation is that, when multiple photoreceptors receive the same stimulus, as will occur in the case of many natural photopic stimuli, independent noise in photoreceptors is reduced through signal averaging and downstream noise in interneuron transmission or ganglion cell spike generation has a greater influence on temporal variability.

A second aspect revealed through the use of ultrasound stimuli involves prolonged dynamics in temporal filtering (FIG. 11). Although ultrasound stimuli are of shorter latency, consistent with stimuli that bypass the phototransduction cascade, they nonetheless have prolonged dynamics. This supports the idea that circuit elements downstream of photoreceptors have a significant influence on temporal filtering, as has been suggested from current injection in inhibitory amacrine cells.

Finally, it was observed that a modulation of visual sensitivity that occurs without a change in temporal filtering, as has been observed from direct current injection into sustained amacrine cells. This supports the idea that the control of sensitivity and temporal filtering are to some extent independent.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1. A retinal stimulation and prosthetic device, comprising:
a. At least one ultrasonic transducer, wherein said at least one ultrasonic transducer comprises a focused ultrasonic signal, wherein said focused ultrasonic signal comprises an acoustic frequency, a spot size, a temporal pattern, a pulse duration and a power capable of stimulating retinal neurons when said at least one ultrasonic transducer is disposed proximal to an eye.

2. The retinal stimulation and prosthetic device of claim 1, wherein said ultrasonic transducer is selected from the group consisting of a planar ultrasonic transducer, a planar ultrasonic transducer array, a 2-D flexible disk ultrasonic transducer, a 2-D flexible disk ultrasonic transducer array, an annular ring ultrasonic transducer, and an annular ring ultrasonic transducer array.

3. The retinal stimulation and prosthetic device of claim 1, wherein said acoustic frequency, said spot size, said temporal pattern, said pulse duration and said power are capable of generating response information necessary for evaluating the health of a retina.

4. The retinal stimulation and prosthetic device of claim 1, wherein said focused ultrasonic signal is capable of focusing at any location of a retina.

5. The retinal stimulation and prosthetic device of claim 1, wherein said at least one ultrasonic transducer is coupled to an optical imaging system, wherein said optical imaging device is capable of imaging a field of view, wherein said optical imaging system is capable of generating imaging signals capable of exciting said at least one transducer to reproduce an image of said field of view, wherein said image of said field of view comprises a radiation pressure to enabling a sensation of vision.

6. The retinal prosthesis of claim 1, wherein said frequency is in a range from 20 MHz to 100 MHz.

7. The retinal prosthesis of claim 1, wherein said spot size is in a range of 150 microns to 15 microns.

8. In another aspect of the invention, the pulse duration is in a range of 0.1 to 50 ms.

9. The retinal prosthesis of claim 1, wherein said power is in a range of 0.1 to 30 W/cm².
